(19) United States
(12) Patent Application Publication
(10) Pub. No.: US 2010/0159539 A1
(43) Pub. Date: Jun. 24, 2010

(54) METHODS AND SYSTEMS FOR PRODUCING BIOFUELS AND BIOENERGY PRODUCTS FROM XENOBIOTIC COMPOUNDS

(76) Inventors: Miguel ASCON, Baltimore, MD (US); Dolores Ascon, Baltimore, MD (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/403,975

(22) Filed: Mar. 13, 2009

Related U.S. Application Data
(60) Provisional application No. 61/069,312, filed on Mar. 13, 2008.

(51) Int. Cl. C12P 7/64; C12M 1/00; C12P 7/06; C12P 7/16; C12P 7/04; C12P 3/00

(52) U.S. Cl. 435/134; 435/289.1; 435/292.1; 435/161; 435/160; 435/157; 435/168

(57) ABSTRACT

The present invention provides methods and systems for producing biofuel and bioenergy products using, as starting raw material, xenobiotic materials or compounds. The xenobiotic materials or compounds may originate from industrial or chemical plants, municipal waste, pharmaceutical products, cosmetic and personal care products, or other sources, and may include aliphatic and aromatic hydrocarbons, chlorinated organic solvents and other halogenated hydrocarbons, as well as heteroaromatic compounds. In accordance with the invention, these materials act as a carbon source to support the metabolism of xenobiotic-degrading microorganisms, thereby producing biomass and/or biogas that may be converted to bioenergy products by microbial synthesis. For example, the biomass may be converted to products such as ethanol, methanol, butanol, and methane, among others. The biogas may be converted to hydrogen gas and biodiesel, among others. Thus, the present invention couples the microbial breakdown (decomposition) of xenobiotic materials with the microbial synthesis of biofuel, thereby supplying needed (inexpensive) energy products, while reducing environmental pollution and contamination, and reducing the costs associated with disposal of hazardous waste.

### PRIORITY

This application claims priority to U.S. Provisional Application No. 61/069/312, filed Mar. 13, 2008, which is hereby incorporated by reference in its entirety.

### FIELD OF THE INVENTION

The present invention relates to the production of biofuels and bioenergy products, including ethanol, butanol, biodiesel, methane, hydrogen, and methanol among others. The present invention relates to the production of such fuels from waste materials, and particularly xenobiotic compounds that may be hazardous or toxic, and which otherwise pollute or contaminate the environment.

### BACKGROUND

There is an ever-increasing demand for renewable biofuels and bioenergy products as an alternative to fossil fuels. Biofuels are currently produced from, for example, food and cellulosic materials, such as agroindustry products, corn, sugar cane, rice, potatoes (among others), as well as wood chips. While the process is straight forward, producing biofuels and bioenergy products from these materials is, overall, inefficient and expensive given the cost of the source materials, and tends to drive up the price of food. Further, the current raw material sources for production of biofuel will not be sufficient to meet the escalating demands.

In the United States, more than four billion pounds of xenobiotic materials are produced, which, after their primary use, are ultimately released into the environment causing significant pollution and contamination. Xenobiotic materials include a variety of industrial and chemical waste products, oil spills, and other toxic substances, including pharmaceuticals, cosmetics, and pesticides. Xenobiotic substances include various synthetic organic compounds that are foreign to the environment and which may persist for long periods of time, and which may be toxic to humans, wildlife, and plant life.

Industry and municipalities spend billions of dollars each year for disposal of hazardous waste, and in fact, the cost to dispose some industry byproducts is greater than the income generated from sales of the corresponding product. These disposal costs represent an overall economic loss, in addition to the negative impact of the pollutants on the environment. Table 1 illustrates some annual hazardous waste disposal (incineration) costs in the United States as reported by the EPA, based on a disposal cost of $3,000 per ton.

TABLE 1

| | Tons | Total |
|---|---|---|
| Industry | | |
| Paper | 105,794 | $317,382,000 |
| Chemical | 262,222 | $786,666,000 |
| Plastic and rubber | 32,023 | $96,069,000 |
| Hazardous waste | 103,631 | $310,893,000 |
| Electric utilities | 514,252 | $1,542,756,000 |
| Textiles | 1,875 | $5,625,000 |
| Major pollutants | | |
| polychlorinated | 749 | $2,247,000 |
| biphenyls | | |
| toluene | 24,403 | $73,209,000 |
| styrene | 25,267 | $75,801,000 |
| Total disposal cost | | $3,210,648,000 |

An integrated system and method for converting xenobiotic materials and compounds to useful products, such as biofuels and bioenergy products, would fulfill a great need, for example, by supplying inexpensive energy, substantially decreasing the cost of disposing hazardous materials, meanwhile reducing the impact of industry on the environment.

### SUMMARY OF THE INVENTION

The present invention provides methods and systems for producing biofuel and bioenergy products using, as starting raw material, xenobiotic materials or compounds. The xenobiotic materials or compounds may originate from industrial or chemical plants, municipal waste, or other sources, and may include aliphatic and aromatic hydrocarbons, chlorinated organic solvents and other halogenated hydrocarbons, as well as heteroaromatic compounds. In accordance with the invention, these materials act as a carbon source to support the metabolism of xenobiotic-degrading microorganisms, thereby producing biomass and/or biogas. The biomass and/or biogas may be converted to bioenergy products by microbial synthesis. For example, the biomass may be converted to products such as ethanol, methanol, butanol, biodiesel, methane, and hydrogen, among others. Thus, the present invention couples the microbial breakdown (decomposition) of xenobiotic materials with the microbial synthesis of biofuel, thereby supplying needed (inexpensive) energy products, while reducing environmental pollution and contamination, and reducing the costs associated with disposal of hazardous waste.

In one aspect, the present invention provides a method for generating one or more biofuels or bioenergy products. The method comprises decomposing xenobiotic materials by microbial action (e.g., biodegradation or decomposition) to produce biomass and/or biogas, coupled with synthesis of one or more biofuels from the biomass and/or biogas by fermentative, methanogenic, and/or photosynthetic microorganisms.

In accordance with this aspect, decomposition of the xenobiotic compound takes place through a plurality of microbial metabolic processes, which may take place in series, or may take place simultaneously in a coupled bioreactor recycling xenobiotic substrate, for example between one or more aerobic and one or more anaerobic bioreactors. For example, xenobiotic waste material may be degraded by circulating xenobiotic-containing liquid material between one or more multiphasic aerobic and one or more multiphasic anaerobic bioreactors, with partially degraded materials, metabolites, cells, and cellular debris circulating through the system(s) until substantial or complete degradation/mineralization of the xenobiotic substrate.

The bioreactors for decomposition (aerobic and anaerobic) may harbor xenobiotic-degrading microorganisms on support surfaces within biofilms. The microorganisms may include pure or mixed cultures of bacteria, yeasts, fungi, and/or algae as described herein. The aerobic and/or the anaerobic bioreactor(s) may be multiphasic bioreactors comprising solid and/or liquid surfaces upon which the xenobiotic-degrading microbes forming biofilms will be active for decomposing the xenobiotic material/compound.

The biomass produced by xenobiotic-degrading microbes is fed, or circulates to, one or more bioreactors for the biosynthesis of bioenergy products including, for example, methane, ethanol, butanol, and methanol, which are recovered and/or purified. For example, ethanol, butanol, or methanol may be produced by fermentation of the biomass by one or more microorganisms such as certain bacteria, yeasts, and filamentous fungi, and the biofuel products subsequently recovered. Methane may be produced by one or more methanogenic microorganisms (such as a microorganism consortium), and recovered and/or purified. Partially degraded xenobiotics, metabolites, cells, cellular debris, and other non-fuel compounds may be subjected to further degradation, for example, by feedback through the degradation system.

CO₂, as may be produced during anaerobic processes, may be used as a carbon source to support the growth and metabolism of photosynthetic microorganisms (e.g., blue-green algae) to synthesize additional biofuels, such as biodiesel (or synthetic intermediates such as lipids) and hydrogen gas.

In a second aspect, the present invention provides systems for generating biofuels and bioenergy products, for example, in accordance with the methods described herein. The system may comprise a biodegradation system, and a separate biosynthesis system. Biomass and/or biogas may be produced with the biodegradation system from xenobiotic substrate, and subsequently fed to the biosynthesis system. Such systems allow for the transportation of biomass and/or biogas produced at one location, to be transported to another for synthesizing biofuel or bioenergy products. Alternatively, the system may be an integrated system for coupling the biodegradation of xenobiotic compounds with the biosynthesis of bioenergy products from resulting biomass and/or biogas. The system (or the biodegradation system) may be connected to, or positioned or located near, the production or source of such xenobiotic compounds, so as to obviate the need to transport the waste, which may be hazardous or toxic, for disposal.

The system comprises one or more bioreactors suitable for decomposing a xenobiotic compound by microbial action, to produce biomass. For example, the one or more bioreactors may be suitable for breaking down toxic material by aerobic and anaerobic processes, including but not limited to a coupled aerobic-anaerobic recycle biofilm reactor, an in-series anaerobic-aerobic biofilm reactor system, or an independent aerobic and anaerobic biofilm reactor system. In accordance with these embodiments, the coupled aerobic-anaerobic biodegradation system allows for the cooperative metabolism of aerobic and anaerobic microorganisms to completely or substantially degrade and mineralize even recalcitrant xenobiotics.

The system further comprises bioreactor(s) suitable for the biosynthesis of biofuels from the biomass. The bioreactor(s) for biosynthesis contain naturally selected or genetically engineered microorganisms for the synthesis of products such as, for example, ethanol, methanol, butanol, and methane from biomass. The biosynthesis reactor(s) may be anaerobic multiphasic bioreactor(s) having fermentative and/or methanogenic microbes supported in biofilms on solid surfaces.

In certain embodiments, the biosynthesis system further comprises at least one photo bioreactor to support the production of additional biofuel products by photosynthetic microorganisms, including one or a consortium of algae(s). The metabolism of the photosynthetic microorganisms is supported by the CO₂ produced during anaerobic decomposition and biosynthesis.

The system may further comprise mechanism(s) for collecting and/or recovering bioenergy products resulting from the degradation or synthesis processes. The system may further comprise a container or feed to recover non-fuel compounds for feedback to the biodegradation system. Thus, the system may comprise a feedback connection between the biosynthesis system and the biodegradation system to continuously recycle all materials not completely used, to avoid the production of virtually any pollutants.

### DESCRIPTION OF THE FIGURES

FIG. 1 diagrams an exemplary coupled or integrated biodegradation/biosynthesis system (BIODSYNT). As shown in FIG. 1, xenobiotic compounds are introduced into a multiphasic bioreactor to degrade the xenobiotic compound. The multiphasic bioreactor for biodegradation may comprise aerobic and anaerobic bioreactors working independently, in-series, or in cycle (a feedback loop). Metabolites and cells from the biodegradation process are fed or circulated to one or more bioreactors for synthesis of biofuels and bioenergy products. The multiphasic bioreactors for biosynthesis may comprise anaerobic bioreactor(s) and photo reactor(s). The biodegradation and the biosynthesis processes/systems are connected by a feedback loop, such that partially degraded products and metabolites are recycled through the system.

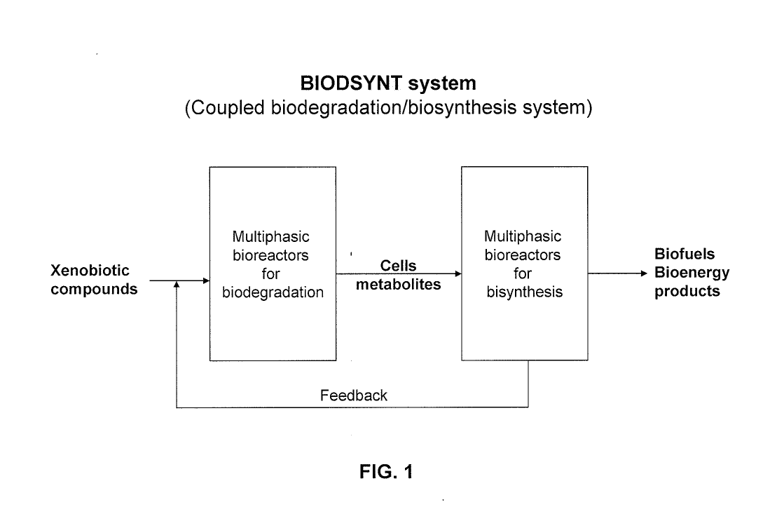

FIG. 2 illustrates an exemplary coupled or integrated biodegradation/biosynthesis system. Xenobiotic compounds are circulated through anerobic (AN)/aerobic (AE) bioreactors working in concert to degrade the xenobiotic material (left side AN and AE reactors). Degraded material (biomass or metabolites) is fed or circulated as liquid to an anaerobic (AN) synthesis bioreactor to produce biofuels and bioenergy products (lower right), with partially degraded material and metabolites recirculating through the system by feedback to the biodegradation systems. Gas produced by the anaerobic processes, such as CO₂, is used to support the metabolism of photosynthetic microorganisms in a photosynthetic reactor (PH). The photosynthesis reactor also produces biofuel and bioenergy products such as biodiesel and hydrogen.

FIG. 3 illustrates an exemplary internal structure of the multiphasic bioreactor system. The multiphasic bioreactors each have solid or liquid surfaces, such as porous glass, silicone rubber, silicone oil, among others, to support microbial biofilms. The bioreactors in this way may maximize the surface areas to support extensive microbial metabolism of xenobiotic compounds. The multiphasic bioreactors may contain, in addition to solid support surfaces, liquid surfaces as well as cellular, aqueous, and gas phases.

FIG. 4 illustrates the internal structure of a multiphasic bioreactor in detail, showing microbial biofilms formed on liquid or support systems, with liquid and/or gas phases.

FIG. 5 shows the production of methane from a toxic and recalcitrant xenobiotic compound, 3,4 dichlorobenzoic acid (3,4,-DCB). As shown, nearly 100% of the xenobiotic was removed from the starting material, with approximately 60% of the xenobiotic mass converted to biogas (e.g., methane) and approximately 40% remaining as biomass. The remaining biomass may act as a substrate for fermentation.

FIG. 6 shows the production of methane from Naproxen. As shown, nearly 100% of the xenobiotic was removed from the starting material, with approximately 50% of the xenobiotic mass converted to biogas (e.g., methane) and approximately 45% remaining as biomass. The remaining biomass may act as a substrate for fermentation.

FIG. 7 shows the production of biogas from the biomass produced from 3,4-DCB and Naproxen biodegradation. An anaerobic biosynthesis bioreactor was fed with the biomass produced in the degradation bioreactors. At 20 hours retention time, the biogas reached 90% with nearly all biomass consumed.

FIG. 8 shows the production of biodiesel from waste CO₂ by algae (at 1% and 0.5% CO₂) as well as the production of hydrogen by algae (at 1% and 0.5% CO₂).

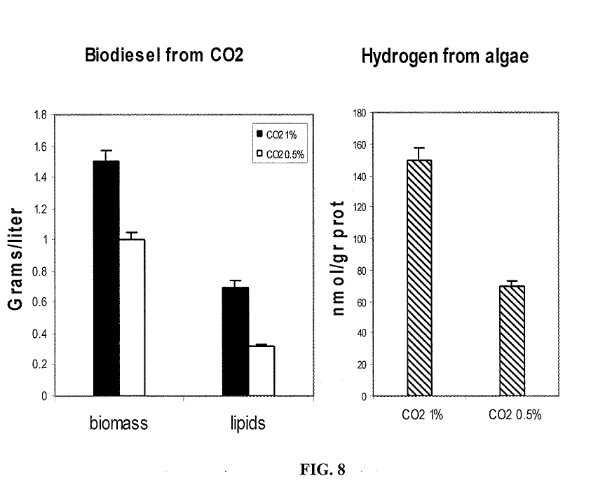

### DETAILED DESCRIPTION OF THE INVENTION

The present invention provides methods and systems for producing biofuel and bioenergy products using, as starting raw material, xenobiotic materials or compounds. The xenobiotic materials or compounds may originate from industrial or chemical plants, municipal waste, or other sources, and may include aliphatic and aromatic hydrocarbons, chlorinated organic solvents and other halogenated hydrocarbons, as well as heteroaromatic compounds. In accordance with the invention, these materials act as a carbon source for the production of energy products including ethanol, methanol, butanol, methane, and hydrogen gas, among others. The present invention couples the breakdown (decomposition) of xenobiotic materials with the synthesis of biofuels, thereby supplying needed (inexpensive) energy products, while reducing environmental pollution and contamination, and reducing the costs for disposal of hazardous waste.

### Xenobiotic Compounds

As used herein, the term “xenobiotic” is a carbonaceous substance produced by man or a carbonaceous metabolite thereof. For example, the xenobiotic may be a pollutant such as a dioxin or polychlorinated biphenyl. In certain embodiments, the xenobiotic is a substance that does not exist in nature. In certain other embodiments, the xenobiotic is a substance found at high concentrations in waste material, so as to be considered hazardous or toxic, such as pharmaceuticals (e.g., antibiotics) and pharmaceutical byproducts, cosmetics, pesticide or fossil fuel pollution. The xenobiotic may be a recalcitrant xenobiotic that is resistant to microbial degradation in the environment (e.g., in water or soil).

The invention may employ any carbonaceous xenobiotic compound (e.g., having carbon in its molecular structure). In accordance with the invention, such compounds act as a carbon source or starting raw material for the production of biofuels and bioenergy products including methane, ethanol, butanol, methanol, biodiesel, and hydrogen, among others.

The xenobiotic material may be one or a combination of aliphatic and/or aromatic hydrocarbons (including polycyclic aromatic hydrocarbons), halogenated organic compounds (including chlorinated organic solvents and chlorinated polycyclic hydrocarbons), and heteroaromatic compounds. The xenobiotic compound may be a polar or hydrophilic compound, and therefore soluble in an aqueous phase. In other embodiments, the xenobiotic is non-polar or hydrophobic, and thus is insoluble in an aqueous phase. The xenobiotic compounds may be one or any combination of organic (solid and liquid) and aqueous materials, and may originate from (without limitation) industrial and/or chemical waste or spills, hydrocarbon waste (e.g., refinery waste, crude oil), agricultural waste or run-off, contaminated or polluted municipal water, biological waste (including infectious waste), food production waste, and household or hospital waste. The xenobiotic material may comprise one or more hazardous or toxic contaminates including organic solvents, pesticides (e.g., DTT), herbicides, fertilizers, plasticizers, dyes, pigments, fire retardants, surfactants, medical and drug by-products including pharmaceuticals as well as synthetic intermediates and metabolites, and cosmetics and personal care products, among others. Generally, the material provided for generation of biomass in accordance with the invention is a suitable carbon source, such as any number of synthetic organic compounds or materials, including aliphatic, aromatic, and/or halogenated hydrocarbons.

Exemplary xenobiotic compounds, which may be converted to biomass include various aliphatic organic molecules, for example, as may originate from petroleum, oil, gasoline, or diesel pollution, among others. Such materials may comprise long or medium chain hydrocarbons, which may be one or a mixture of saturated and/or unsaturated molecules, and one or a mixture of substituted and/or unsubstituted molecules, as well as one or a mixture of linear or cyclic molecules. The aliphatic hydrocarbons may include those having a hydrocarbon chain of from 6-44 carbon atoms, or in certain embodiments, from 4-20 carbon atoms. For example, the xenobiotic material may comprise a C5 to C16 saturated hydrocarbon, such as an alkane (e.g., cyclohexane, octane, decane). In some embodiments, the xenobiotic material comprises an unsaturated hydrocarbon, such as an alkene (e.g., ethylene). Exemplary substituents include alkyl (methyl, ethyl, isobutyl, etc.), halogen (e.g, Cl, Br, F, and I), hydroxy, amine, amide, etc. The xenobiotic material may comprise a halogenated hydrocarbon (e.g., chlorinated hydrocarbon), such as trichloroethylene, tetrachloroethylene, perchloroethylene, and 1,1,1-trichloroethane. In certain embodiments, the xenobiotic material comprises an ether (e.g., methyl tertiary butyl ether) or aldehyde (e.g., acetyl aldehyde, formaldehyde). In some embodiments, the xenobiotic hydrocarbon is cyclic (e.g., cyclohexane, hexachloro cyclohexane).

The xenobiotic material may be a compound having one, two, three, or more aromatic systems (e.g., polyaromatic hydrocarbon), which may each independently include one, two, or three heteroatoms (e.g. N, O, and/or S). In certain embodiments, the xenobiotic material comprises a nitroaromatic compound. Such aromatic systems may each independently contain from 5 to 20 carbon atoms, including aromatic systems of from 6 to 12 carbon atoms. Two or more aromatic systems may be fused, and with respect to 6-membered rings may be substituted at ortho-, para-, and meta-positions (e.g., with a halogen, hydroxy, alkyl, and/or nitro group, among others). Exemplary xenobiotics in accordance with these embodiments include benzene, methyl benzene, dimethyl benzene, ethyl benzene, phenol, methyl phenol, and polycyclic aromatic hydrocarbons such as naphthalene and polychlorinated biphenyls (PCBs such as trichlorophenyl).

In these or other embodiments, the xenobiotic material may comprise at least one compound having at least one carbon-halogen bond, including with Cl, Br, FI, and/or I. For example, a xenobiotic compound may be mono-, di- or tri-halogenated. Exemplary compounds include polychlorinated diaromatic hydrocarbons (PCDH). The compound may be a dioxin. Exemplary xenobiotic compounds in accordance with these embodiments include methylchloride, methylene chloride, dichloroethylene, chloroform, chloroacetic acid, dichloromethane, dichloroethane (e.g., 1,2-dichloroethane), 1,1,1-trichloroethane, toluene, xylene, vinyl chloride, 1,3-dichloropropylene, 1,2-dichloropropane, 1,2,3-trichloropropane chlorobenzene, hexachlorobenzene, trichlorobenzene (e.g., 1,2,4-; 1,2,3-; or 1,3,5-), hexachlorobutadiene; styrene (e.g., octachlorostyrene), chlorinated furan, epichlorohydrin, hexachlorocyclohexane, trichloroethylene, pentachlorophenol, 2-chloropropionate, 1-chlorobutane, and atrazin. Others include 1,2-dibromomethane, 1,2-dibromoethane, 2,4,6-tribromophenol, fluoroethanes, fluoropropanes, and iodoisophthalic acid.

Depending on the characteristics of the source material and bioreactor system, xenobiotic materials may be pretreated before biodegradation to purify/isolate or concentrate these components from large volumes of waste material, and/or to remove undesired matter or impurities. Where the xenobiotic is solid waste (e.g., plastics, papers, polystyrene), the xenobiotic may be first hydrolyzed, e.g., by acid or base hydrolysis, to modify the macromolecular components and render them essentially soluble in aqueous or organic phase. In certain embodiments, materials that are potentially toxic to microorganisms at high concentration, such as heavy metals if present, may be removed or sufficiently diluted.

While xenobiotic compounds vary significantly in their ability to persist in the environment due to the stabilities of various chemical bonds to microbial digestion, in accordance with various embodiments of the invention, such compounds and materials can be efficiently and substantially decomposed, thereby achieving substantial or complete mineralization of otherwise toxic materials, while converting suitable organic material to useful biofuel products. Further, while bioremediation of xenobiotics can itself produce substances that are pollutants or are toxic to animal and plant life, the present invention provides methods and systems for preventing accumulation of virtually any significant pollutants (a “zero pollution system”) while achieving substantially complete mineralization with biofuel production.

### Basic Media

In certain embodiments, xenobiotic effluents from industrial discharges will have an aqueous phase containing some inorganic nutrients capable of supporting microbial growth and metabolism. However, where these inorganic nutrients are absent, or are present in insufficient amounts, the xenobiotic-containing material may be supplemented with an aqueous phase containing a basic mineral salt medium to support microbial growth and metabolism of xenobiotic-degrading microorganisms.

In the bioreactors, the aqueous phase(s) comprise inorganic nutrients to support microbial growth and metabolism. For example, the aqueous phase may be a mineral salts medium. Nitrogen and phosphorous are the main nutrients added to the aqueous phase. Micronutrients such as Ca, Zn, Mn, Cu, Fe, Mg, Mn, Mb, and S may also be present in at least trace amounts. An exemplary mineral salts medium is KHCO₃ (e.g., 2 g/L), NaHCO₃ (e.g., 1.8 g/L), KH₂PO₄ (e.g., 0.7 g/L), Na₂HPO₄.12H₂O (1.4 g/L), MgSO₄. 7H₂O (e.g., 0.2 g/L), and (NH₄)₂SO₄ (e.g., 0.8 g/L). The medium may further contain trace elements as may be required to support growth and vitality of the microorganisms, such as Ca(H₂PO₄) (e.g., 40 mg/L), ZnSO₄.7H₂O (5 mg/L), Na₂MoO₄.2H₂O (2.5 mg/L), FeSO₄. 7H₂O (1 mg/L), MnSO₄.H₂O (1 mg/L), and CuSO₄ (0.6 mg/L). The nutrient medium may of course be adjusted based upon the metabolic requirements of the microorganism(s) selected for decomposition and biosynthesis.

### Bioreactors

The invention involves feeding the xenobiotic compound or material into a bioreactor system. The system generally comprises bioreactors for biodegradation that will produce (e.g., in batch, semicontinuously, or continuously) biomass and/or biogas, as well bioreactors for the synthesis of biofuels from the resulting biomass and/or biogas. The degradation/synthesis bioreactors may be coupled to provide an integrated system. As used herein, the term “biomass” refers to liquid or sludge containing decomposed xenobiotic compound, metabolites, microbial cells, and cellular debris. The biomass produced by decomposition of the xenobiotic acts as a substrate for the synthesis of biofuels and bioenergy products. “Biogas” refers to gaseous products of microbial metabolism, and includes largely carbon dioxide and methane.

In various embodiments, the present invention involves bioreactors to decompose xenobiotic materials by the combined metabolism of aerobic and anaerobic microorganisms, as well as one or more bioreactors to support the synthesis of one or more biofuels from the biomass and/or biogas by fermentative, methanogenic, and/or photosynthetic microorganisms.

The type of bioreactors and internal designs may be selected on the basis of, for example, desired volume and/or retention time, solubility or insolubility of the xenobiotic compound in an aqueous phase, the level of toxicity of the xenobiotic to degrading microbes, the selection of microorganisms for degradation, which as described below may include bacteria, yeasts, fungi, and algae, and may include aerobic and anaerobic environments. Additional factors that may be considered in selecting the appropriate reactors include the mechanisms of xenobiotic uptake by the microbes, microbial oxygen demand, and desired flow and agitation system.

For example, where the xenobiotic compound is sufficiently water soluble and substantially non-toxic to degrading microbes, the bioreactor for decomposition may comprise a large aqueous phase. Alternatively, where the xenobiotic is fairly insoluble in an aqueous phase and/or is toxic to degrading microorganisms, the reactor for decomposition may further comprise heterogenic liquid and/or solid phases.

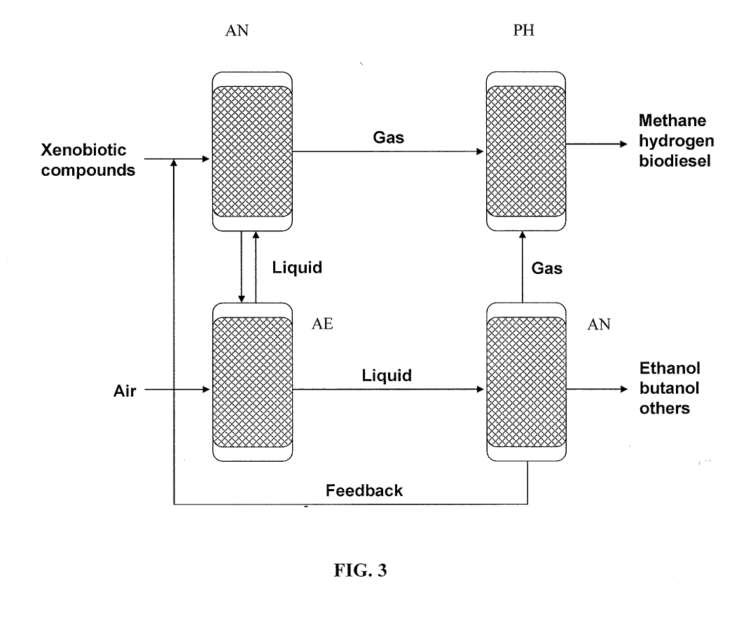

The bioreactors may harness microbes in aqueous suspension and/or supported on surfaces. For example, the bioreactors may comprise solid surfaces that support xenobiotic-degrading microbes or biofuel-synthesizing microbes within biofilms. The solid surfaces may be composed of a variety of materials including porous glass, silicone rubber, as well as polymeric or metal surfaces (for example). The solid surfaces may form a fixed-bed reactor, that is, via a fixed solid support matrix (see FIG. 3). See also, Ascon-Cabrera et al., Activity of Synchronized Cells of a Steady-State Biofilm Recirculated Reactor During Xenobiotic Biodegradation Appl. Environ. Microbiol. Vol. 61(3)920-925 (1995). Alternatively or in addition, one or more support surfaces may be in the form of polymeric beads and the like, which may form a support bed or support matrix.

The bioreactors may be multiphasic reactors having solid phases (support surfaces and microbial cells), liquid phases (aqueous and/or organic phases), and gas phases (air, and gas produced by microbial metabolism). Aqueous liquid and gas phases may circulate within or between the various reactors as described herein. Where aqueous and organic (oil) phases are employed, microbes may form biofilms at the liquid interface as well as on the solid support surfaces. For example, silicone oils (e.g., polydimethylsiloxane) may be used as the organic phase for poorly water-soluble xenobiotic compounds. Additional exemplary solvents, suitable for poorly soluble xenobiotic compounds are known.

Where aqueous-organic interfaces are employed the xenobiotic substrate diffuses from the organic phase to the aqueous phase. In this system, the microorganisms forming a biofilm will carry out substrate conversion in the interfacial area and/or aqueous phase, while the metabolites that have low aqueous solubility will be extracted by the organic phase. See, Ascon-Cabrera et al., Interfacial area effects of a biphasic aqueous/organic system on growth kinetics of xenobiotic-degrading microorganisms, Appl Microbiol. Biotechnol. 43:1136-1141 (1995).

The bioreactor system may be a batch system, a semi-continuous system, or may be a continuous system, such as a recirculated biofilm reactor. In such systems, a steady-state biofilm is produced and maintained. The steady state may be a balance of several factors, for example, cell processes of attachment, shear stress, limitations of substrate concentration, and cell growth rate, as well as the physiological state of the cells. In certain embodiments, the bioreactor system is a continuous-flow fixed-bed reactor system, for example, as described in Ascon-Cabrera et al., Activity of Synchronized Cells of a Steady-State Biofilm Recirculated Reactor During Xenobiotic Biodegradation, Appl. Environ. Microbiol. 61(3):920-925 (1995).

In certain embodiments, the bioreactor for decomposition of the xenobiotic comprises one or more aerobic (AE) and one or more anaerobic (AN) bioreactors, which may be continuous, semi-continuous, or batch systems, and which each be fixed-bed reactor systems and/or multiphasic reactor systems as described. The aerobic bioreactor(s) harbor aerobic and/or facultative microorganisms (e.g., bacteria) with sufficient air (including oxygen) for supporting aerobic metabolism. In contrast, the anaerobic component harbors anaerobic or facultative microorganisms with a sufficient absence of air (oxygen) for supporting anaerobic metabolism. In certain embodiments, the anaerobic bioreactor is anoxic or has anoxic regions, which have a substantial absence of oxygen. The cooperative aerobic and anaerobic metabolisms generally promote complete mineralization of the xenobiotic with optimal biomass production.

There are several ways for integrating aerobic and anaerobic processes, each of which may be employed in connection with the invention. For example, aerobic and anaerobic processes may be integrated by co-culture of aerobic and anaerobic microorganisms in chemostats under microaerophilic conditions or with oxygen gradients, co-immobilization of aerobic and anaerobic microorganisms on gel or other supports, the culture of mixed aerobic-anaerobic microorganisms in conditions changing from anaerobic to aerobic (e.g., including by oxygen gradients), aerobic and anaerobic reactors connected in series, and recycling connected aerobic and anaerobic reactors. An exemplary anaerobic and aerobic integrated reactor is disclosed in U.S. Pat. No. 5,599,451, which is hereby incorporated by reference in its entirety.

In some embodiments, the bioreactor for decomposition comprises a coupled aerobic-anaerobic recycle biofilm reactor system (CAR), an in-series anaerobic-aerobic biofilm reactor (SAR) system, or an independent aerobic and anaerobic biofilm reactor (IAR) system. Such systems are described, for example, in Ascon et al., High efficiency of a coupled aerobic-anaerobic recycling biofilm reactor system in the degradation of recalcitrant chloroaromatic xenobiotic compounds, Appl. Microbiol. Biotechnol. 52:592-599 (1999). In some embodiments, the invention employs a CAR system, to allow for a cooperative metabolism between aerobic and anaerobic bacteria (caused by an exchange of cells and metabolites between AE and AN reactors). Such systems can overcome the metabolic and kinetic limitations of aerobic and anaerobic bacteria in uncoupled AE and AN reactors. Generally, in such systems, aerobic and anaerobic biodegradation processes are used to assure a total degradation of recalcitrant xenobiotics, such as haloaromatics and other halogenated hydrocarbons. For example, chlorinated compounds may be more rapidly dechlorinated by anaerobic microorganisms, while dechlorinated compounds can be completely mineralized by aerobic microorganisms.

The bioreactor system for biosynthesis comprises at least one bioreactor that is suitable for fermentation of the biomass and/or methanogenesis. The bioreactor for fermentation (e.g., for production of alcohols) and/or methanogenesis is generally one or more anaerobic bioreactor(s), such as a multiphasic anaerobic bioreactor. The reactor conditions such as temperature and level of oxygenation and aeration (for example) for fermentation and methanogenesis are well known, and may be adjusted depending on the microorganisms employed in the certain embodiments. Such conditions are further described in U.S. Pat. No. 6,555,350, U.S. Pat. No. 7,354,743, U.S. Pat. No. 7,498,163, U.S. Pat. No. 7,351,559, U.S. Pat. No. 7,455,997. These patents are hereby incorporated by reference in their entireties.

The bioreactor for synthesis of biofuels such as ethanol, butanol, and methanol, may be a biofilm multiphasic anaerobic reactor(s). These reactors contain microorganisms (in pure or mixed cultures) selected to synthesize the specific biofuel or bioenergy product(s) desired. For example, if the production is ethanol, the microorganism for biosynthesis may be Saccharomyces sp., or Zymomonas, sp. For butanol and methanol production, the microorganism may be Clostridium sp. or Methanomonas sp., respectively. The reactor conditions such as temperature, oxidation-reduction conditions, pH, fed and recirculation rates, etc. may be adjusted depending on the microorganisms, carbon source, reactors characteristics, among others, employed in certain embodiments.

The bioreactor system for photosynthesis may likewise be a multiphasic system containing photosynthetic microorganisms such as blue green algae and others described herein, which may be supported by effluent CO₂ from the anaerobic bioreactors. Photo reactors are known, such as those described in U.S. Pat. No. 7,371,560, which is hereby incorporated by reference in its entirety. The aeration, temperature, pH, nutritional requirements, intensity and wavelength of light (e.g., white light), as well as duration of light/dark cycles suitable for photosynthetic microorganisms are known, for example, as also described in U.S. Pat. No. 7,371,560.

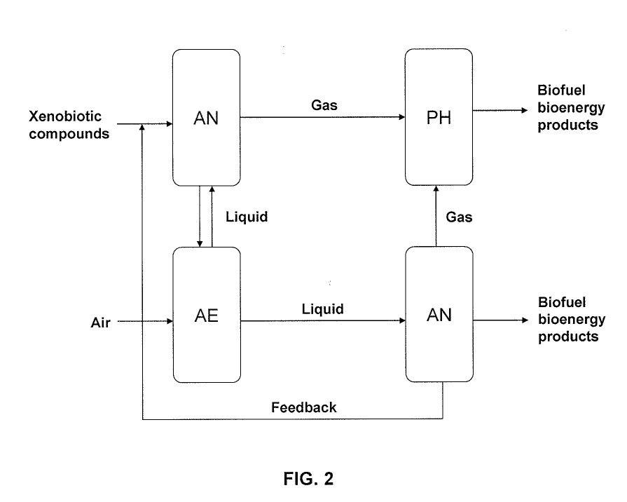

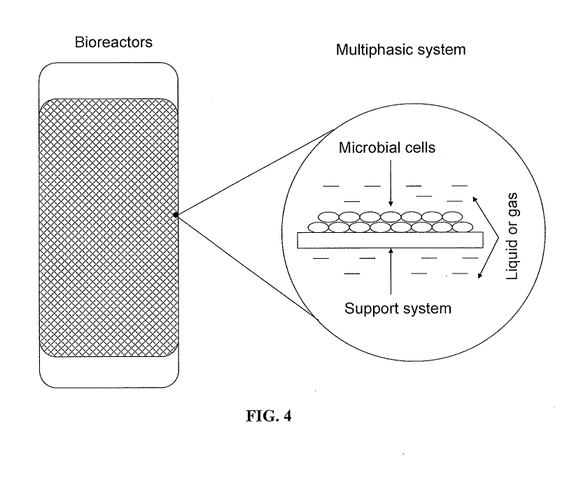

The method may employ a multiphasic, coupled, reactor system as described in more detail herein, and as illustrated in FIG. 2, FIG. 3, and FIG. 4. This system allows for liquid substrate to circulate between the anaerobic and aerobic decomposition reactors. Material from the decomposition reactors also circulates to an anaerobic biosynthesis reactor and comprises a feedback port for feeding recycled, partially metabolized compounds from the biosynthesis reactor(s) back for further biodegradation. The flow rate of material through the system can be controlled by the operation of a system of pumps, valves, etc. A photoreactor is integrated by feeding effluent CO₂ from the anaerobic reactors.

### Microorganisms

Microorganisms suitable for decomposition of xenobiotic compounds, as well as for biofuel and bioenergy product synthesis are known. Exemplary microorganisms are listed in Tables 2 and 3 (below).

The microorganisms for aerobic and anaerobic decomposition of xenobiotic material may be chosen in accordance with the chemical makeup of the xenobiotic, or the estimated or quantified chemical makeup of the mixture. The selection of microorganism(s) may include one or a combination (a consortium) of bacteria, fungi, yeasts, and algae. The selection may take into account such factors as the xenobiotic's identity as, for example, aliphatic hydrocarbon, aromatic hydrocarbon, polycyclic aromatic hydrocarbon, halogenated hydrocarbon, aromatic amine, substituted aromatic (e.g., alkyl, hydroxy, or halogen substituted), heteroaromatic (N, S, and/or O), and/or polychlorinated biphenyl. Such selections may be further guided by: Aust et al., Biodegradation of Hazardous Wastes, Environmental Health Perspectives Supplements Vol. 102 Suppl. 1:245-252 (1994); Jain et al, Microbial Diversity: Application of microorganisms for the biodegradation of xenobiotics, Current Science Vol. 89. No. 1 (2005); Janssen et al., Bacterial degradation of xenobiotic compounds: evolution and distribution of novel enzyme activities, Environmental Microbiology 7(12)1868-1862 (2005); among others.

The microorganism(s) may be selected by known protocols for their degrading activity against a particular xenobiotic (e.g., see Ascon-Cabrera et al., Selection of Xenobiotic-Degrading Microorganisms in a Biphasic Aqueous-Organic System, Appl. Environ. Microbiol. 59(6):1717-1724 (1993). Alternatively, microorganisms may be genetically engineered to express enzymes suitable for degrading the selected xenobiotic and/or other gene products that enhance growth or metabolism in the bioreactors. Techniques for genetic manipulation are well known, and include introduction of extrachromosomal elements by plasmid or phage, or integration of such elements into the host genome. Standard recombinant DNA and molecular cloning techniques are well known in the art and are described for example in Sambrook, J., Fritsch, E. F, and Maniatis, T. Molecular Cloning: A Laboratory Manual; Cold Spring Harbor Laboratory Press: Cold Spring Harbor, 1989. Xenobiotic-degrading microorganisms having well know tools for genetic manipulation include Pseudomonas, Rhodococcus, Bacillus, E. coli, Saccharomyces cerevisiae, among others. Generally, the enzymes introduced will include known dioxygenases for aerobic conditions, reductases for anaerobic conditions, and hydroxylases for facultative conditions (e.g., both aerobic and anaerobic).

The microorganisms for biodegradation may, for example, include one or a consortium of Pseudomonas sp., Arthrobacter sp., Acynebacter sp., and Alcaligenes sp., among others. Exemplary microorganisms known to have xenobiotic-degrading activity are provided below (Table 2), together with exemplary xenobiotics and enzymes involved in decomposition. The conditions (e.g., aerobic, anaerobic, anoxic, facultative) under which each microorganism is metabolically active is also known.

The main enzymes involved in xenobiotic break down are the mono-oxygenases, di-oxygenases, reductases, dehalogenases, laccases, peroxidases, and phosphotriesterases. Examples of enzymes for degrading specific xenobiotic compounds are listed below in the (Table 2). These enzymes may be genetically engineered to increase the degradation performance.

TABLE 2
Microorganisms and Enzymes For Decomposition of Xenobiotics
Aliphatic Hydrocarbons

| | |
|---|---|
| Exemplary | petroleum, cyclohexane, octane, decane, ethylene, methyl tertiary butyl ether |
| xenobiotics | |
| Enzymes | alk-BAC operon (alkane hydroxylase, alcohol dehydrogenase, aldehyde |
| involved | dehydrogenase, rubedoxin(s) (e.g., AlkG)) |
| | AlkB, AlkM |
| Organisms | Pseudomonas sp. (P. oleovorans, P. putida, P. aeruginosa, P. citronellolis, P. |
| | aerofaciens) |
| | Rhodococcus sp. (R. rhodochrous, R. erythropolis) |
| | Acinetobacter sp. (strain ADP1) |
| | Alcaligenes sp. |
| | Burkholderia sp. (B. cepacia RR10 |
| | Arthrobacter sp. |
| | Flavobacterium sp. |
| | Bacillus sp. |
| | Brevibacterium sp. (B. erythrogenes) |
| | Alcanivorax borkumensis |
| | Saccharomyces cerevisiae |

Halogenated Compounds and Chlorinated Solvents

| | |
|---|---|
| Exemplary | methylchloride, methylene chloride, dichloroethylene, ethylene dibromide, |
| xenobiotics | chloroform, chloroacetic acid, 1,2-dichloroethane, 1,2-dibromoethane, 1,1,1- |
| | trichloroethane, vinyl chloride, 1,3-dichloropropylene, 1,2-dichloropropane, 1- |
| | chlorobutane, 2-chloropropionate, 1,2,3-trichloropropane, chlorobenzene, |
| | epichlorohydrin, hexachlorocyclohexane, trichloroethylene, pentachlorophenol |
| Enzymes | haloalkane dehalogenases (DhlA, DhaA, LinB) |
| involved | haloacid dehalogenases (HAD superfamily of hydrolases) (DhlB, HAD-Ps) |
| | halohydrin dehalogenases (HheA, HheB, HheC) |
| | Chloroacrylic acid dehalogenases (CaaD) |
| | dichloromethane dehalogenase (DcmA) |
| | 1,2-dichloroethane dehalogenase |
| | LinB |
| | hydrolytic dehalogenase (CbzA, AtzA) |
| | tetrachlorohydroquinone dehalogenase (PcpC) |
| | haloacetate dehalogenase (dehH2) |
| | monooxygenases and dioxygenases |
| Exemplary | Rhodococcus sp. (R. erythropolis, R. rhodochrous NCIMB 13064) |
| Organisms | Sphingbium chlorophenolicum |
| | Moraxella sp. (strain B) |
| | Pseudomonas sp. (P. pavonaceae, Pseudomonas ADP, P. stutzeri, P. putida, |
| | P.seudomonas Strain Ps1) |
| | Xanthobacter sp. (X. autotrophicus GJ10, X. flavus |
| | Corynebacterium sp. strain m15 |
| | Ancylobacter aquaticus |
| | Arthrobacter sp. |
| | Nitrosomonas europea |
| | Alcaligenes sp. |
| | Ralstonia sp. |
| | Methylosinus trichosporum OB3b |
| | Methylosporovibrio methanica 812 |
| | Methanothrix sp. |
| | Methanosarcina sp. |
| | Mycobacterium sp. strain GP1 |
| | Agrobacterium sp. |
| | Micrococcus sp. |
| | Candida sp. |
| | Trichosporon sp. |

Aromatic Hydrocarbons and Polycyclic Aromatic Hydrocarbons

| | |
|---|---|
| Exemplary | phenol, naphthalene, pentachlorophenol |
| xenobiotics | alkyl benzene (toluene, xylene, ethylbenzene, isobutylbenzene, dibenzofurans) |
| | chlorinated aliphatic hydrocarbons (TCE) |
| | N-heterocyclic aromatic |
| | pyrene |
| | Chlorinated dioxin |
| Enzymes | monooxygenase systems |
| involved | PCP-4 monooxygenase |
| | lignin peroxidase |
| Exemplary | Pseudomonas sp. (P. stutzeri, P. putida, P. cepacia G4, P. mendocina KR, P. |
| Organisms | picketti PK01, P. fluorescens, P. vesicularis, P. paucimobilis, Psuedomonas sp. |
| | DLC-P11, P. mendocina, P. cichhori, strain IST 103) |
| | Rhodococcus sp. |
| | Corynebacterium renale |
| | E. coli |
| | Serratia sp. |
| | Bacillus cereus |
| | Micrococcus sp. (Micrococcus diversus) |
| | Deinococcus radiophilus |
| | Alloiococcus otitis |
| | Acinetobacter sp.(A. calcoaceticus) |
| | Arthrobacter sulfureus |
| | Acidovorax delafieldi P4-1 |
| | Brevibacterium sp. HL4 |
| | Nitrosomonas europaea |
| | Methylosinus trichosporium OB3b |
| | Methylosporovibrio methanico |
| | Methanospirilium |
| | Alcaligens sp. (A. denitrificans) |
| | Mycobacterium sp. |
| | Moraxella sp. |
| | Beijerinckia sp. |
| | Neptunomonas naphthovorans |
| | Sphingomonas sp. (S. yanoikuyae, S. sp. RW1) |
| | Actinomycetes |
| | Klebsiella pneumonia |
| | Phanerochaete chrysosporium (white rot fungus) |

Chlorinated Polycyclic Hydrocarbons

| | |
|---|---|
| Exemplary | Polychlorinated biphenyls (PCBs) |
| xenobiotics | DTT |
| Enzymes | lignin peroxidase, Mn-dependent peroxidase, Mn-independent peroxidase, laccase |
| involved | |
| Exemplary | strain DCB1 |
| Organisms | Acinetobacter sp. |
| | Alcaligenes sp. |
| | Klebsiella pneumoniae |
| | Pseudomonas sp. (P. cruciviae) |
| | White rot fungus (Phanerochaete chrysosporium, Trametes versicolor, Pleurotus |
| | ostreatus) |

Nitroaromatic compounds

| | |
|---|---|
| Exemplary | parathion, methyl parathion, dinoseb, dinitrocresol, nitrofen, |
| xenobiotics | nitrobenzene, dinitrotoluene, dinitrophenol, o-nitrobenzoate, p-nitrophenol, 4- |
| | nitrocatechol |
| Enzymes | oxygenase-based pathways (mono- and di-oxygenases, dehydrogenases) |
| involved | |
| Exemplary | Pseudomonas sp. |
| Organisms | Nocardia sp. |
| | Arthrobacter sp. (A. protophormiae) |
| | Burkholderia cepacia |

Organisms for methanogenesis or fermentation of the biomass may be selected on the basis of the desired product. In certain embodiments, the desired product(s) include one or more of methane, ethanol, and butanol. For the production of methane or low molecular weight alcohols, the microbial population and operating conditions are selected to promote the conversion of organic xenobiotic compounds to volatile fatty acids or low molecular weight (one, two, three, or four carbon) molecules.

The microorganisms for biofuel synthesis may, for example, include one or a consortium of bacteria as methanogens, yeasts such as Saccharomyces sp., anaerobic bacteria such as Clostridium sp., or microalgae such as Chlorella sp. or Synechococcus sp., among others. Exemplary microorganisms having known biosynthesis activity are provided below (Table 3), together with exemplary enzymes involved in biosynthesis pathways. Examples of enzymes for synthesizing particular biofuel compounds are also provided. These enzymes may be genetically engineered to increase their performance.

TABLE 3
Microorganisms and Enzymes for Biosynthesis of Biofuels and Bioenergy Products
Methane

| | |
|---|---|
| Exemplary | Organic acids, CO2 |
| carbon sources | |
| Enzymes | formylmethanofuran dehydrogenase, |
| involved | methyltetrahydro-methanopterin: coenzyme M methyltransferase (Mtr), |
| | heterodisulfide reductase (Hdr), |
| | F₄₂₀H₂ oxidase (FprA), |
| | formaldehyde activating enzyme (Fae) |
| | methenyltetrahydromethanopterin cyclohydrolase, |
| | methylenetetrahydromethanopterin reductase, |
| Exemplary | Methanococcus sp, |
| Organisms | Methanomicrobium sp. |
| | Methanospirilliam sp. |
| | Methanoplanus sp. |
| | Methanosphaera sp. |
| | Methanolobus sp. |
| | Methanoculleus sp. |
| | Methanosaeta sp. |
| | Methanopyrus sp. |
| | Methanocorpusculum sp. |
| | Methanosarcina |

Ethanol

| | |
|---|---|
| Exemplary | Biomass, and carbonic metabolites produced after xenobiotic biodegradation |
| carbon sources | |
| Enzymes | Alcohol dehydrogenase (A, B, and C) |
| involved | Acetaldehyde dehydrogenase |
| | Amylases |
| | Glucoamylases |
| | Invertases |
| | Lactases |
| | Cellulases |
| | Hemicellulases |
| Exemplary | Saccharomyces sp. |
| Organisms | Klyveromyces sp. |
| | Zymomonas sp. |

Butanol

| | |
|---|---|
| Exemplary | Biomass, and carbonic metabolites produced after xenobiotic biodegradation |
| carbon sources | |
| Enzymes | Acetyl-CoAacetyltransferase |
| involved | Acetoacetyl-CoAthiolase |
| | 3-hydroxybutyryl-CoAdehydrogenase |
| | Crotonase |
| | Butyryl-CoAdehydrogenase |
| | Aldehyde/alcohol dehydrogenase |
| Exemplary | Clostridium sp. |
| Organisms | |

Hydrogen

| | |
|---|---|
| Exemplary | Water and light |
| sources | |
| Enzymes | Hydrogenases |
| involved | |
| Exemplary | Clostridium sp. |
| Organisms | |

Biodiesel

| | |
|---|---|
| Exemplary | CO2, and light |
| carbon source | |
| Enzymes | Lipasas (Triacylglycerolhydrolases) |
| involved | |
| Exemplary | Chlorella sp. |
| Organisms | Synechococcus sp. |
| | Synechocystis sp. |
| | Nitzchia sp. |
| | Schizochytriu sp. |

Methanol

| | |
|---|---|
| Exemplary | Methane and oxygen |
| carbon source | |
| Enzymes | Methane monooxygenase |
| involved | Formate dehydrogenase |
| | Formaldehyde dehydrogenase |
| Exemplary | Methylomonas sp. |
| Organisms | Methylosinus sp. |
| | Methylococcus sp. |

Where methane is a desired biofuel product, at least one bioreactor for biosynthesis is an anaerobic reactor that comprises a methanogenic microorganism, which may include one or a consortium of Methanobacterium sp., Methanothrix sp., Methanosarcina sp., and Methanomonas sp. Other methanogenic microbes that may be used, and are described in U.S. Pat. No. 6,555,350, which is hereby incorporated by reference. For example, methanogens also include Methanococcus sp, Methanomicrobium sp., Methanospirilliam sp., Methanoplanus sp., Methanosphaera sp., Methanolobus sp., Methanoculleus sp., Methanosaeta sp., Methanopyrus sp., and/or Methanocorpusculum sp.

Some methanogenic species are highly thermophilic and thus can grow at temperatures in excess of 100° C. Where the methanogen is highly thermophilic, a separate bioreactor for synthesis of methane may be preferred. In certain embodiments, the methanogen is one or a consortium of Methanosarcina, Methanosaeta and/or Methanothrix species, which may carry out conversion of acetate and similar small-molecule carbon substrates to methane and carbon dioxide. Methanogens may use small organic compounds as substrate, such as formic acid (formate), methanol, methylamines, dimethyl sulfide, and methanethiol, which may be produced in the system.

The methanogenic bacteria may be naturally-selected for high methane producing activity, or alternatively, may be genetically modified by known techniques. The methanogenic enzymes that may be genetically engineered include, formylmethanofuran dehydrogenase, methyltetrahydro-methanopterin: coenzyme M methyltransferase (Mtr), heterodisulfide reductase (Hdr), F₄₂₀H₂ oxidase (FprA), formaldehyde activating enzyme (Fae), methenyltetrahydromethanopterin cyclohydrolase, and methylenetetrahydromethanopterin reductase. These enzymes have been isolated from species including, Methanococcus, Methanothermobacter, methanosarcina, methanopyrus, among others.

Methane may also exist as a component of the biogas produced during anaerobic decomposition of the xenobiotic compound. “Biogas” is a product of anaerobic digestion. In the absence of oxygen, anaerobic bacteria decompose organic matter and produce a gas mainly composed of methane (about 60%) and carbon dioxide. This gas can be compared to natural gas, which is approximately 99% methane. Biogas can be collected and used as an energy source for generators, boilers, burners, dryers or any equipment using propane, gas or diesel. Alternatively, methane may be recovered from the biogas as described in greater detail below.

The process and systems described herein may be designed to produce alcohols, such as alcohols containing one to nine carbon atoms. Particular examples of alcohols that can be produced according to the invention include propanol, butanol, pentanol, hexanol, heptanol, octanol and nonanol. For the synthesis of alcohols, particularly ethanol, at least one bioreactor is an anaerobic reactor comprising fermentative microorganisms, such as one or more Zymomonas sp. and/or Saccharomyces sp. (e.g., Saccharomyces cerevisiae) Additional microorganisms suitable for fermentation of the biomass include a number of yeasts such as Klyveromyces sp., Candida sp., Pichia sp., Brettanomyces sp., and Hansenula sp. and Pachysolen sp. Alternatively, the microorganism may be one or a consortium of bacterial species such as Leuconostoc sp., Enterobacter sp., Klebsiella sp., Erwinia sp., Serratia sp., Lactobacillus sp., Lactococcus sp., Pediococcus sp., Clostridium sp., Acetobacter sp., Gluconobacter sp., Aspergillus sp., and Propionibactedum sp.

Various organisms for the production of fermentation products are known, as well as conditions for growth and substrate requirements, and are described for example, in U.S. Pat. No. 7,455,997, U.S. Pat. No. 7,351,559, U.S. Pat. No. 6,555,350, and U.S. Pat. No. 7,354,743, which descriptions are hereby incorporated by reference. In certain embodiments, the desired product is butanol, and the fermentative microorganisms include one or a consortium of bacteria including Clostridium sp.

The fermentative microorganisms, for example to produce ethanol, may be naturally selected for the production of the desired product, or may be genetically engineered to express desired enzymes. Techniques for genetic manipulation of bacteria and yeasts are well known, and include introduction of extrachromosomal elements by plasmid or phage, or integration of such elements into the host genome. Exemplary enzymes that may be genetically engineered include, Alcohol dehydrogenase (A, B, and C), Acetaldehyde dehydrogenase, Amylases, Glucoamylases, Invertases, Lactases, Cellulases, and Hemicellulases, among others.

In certain embodiments, the process and systems described herein will include a photo reactor to convert CO₂ produced during anaerobic xenobiotic decomposition and biofuel synthesis to biofuel products such as hydrogen gas and lipids. Lipids may be employed in the production of biodiesels, for example, by transesterification. Photosynthetic organisms for use in the production of hydrogen gas and lipids from CO₂ are known, and include one or a consortium of naturally selected or genetically modified Synechococcus sp., Chlorella sp., Synechocystis sp., Nitzchia sp., and/or Schizochytriu sp., among others.

For the production of hydrogen, the method and system may employ photosynthetic microorganisms capable of using water as an indirect substrate for hydrogen production. Such microorganisms generally express one or more hydrogenases. These may include cyanobacteria and algae, such as green algae, blue-green algae, or red algae. Exemplary species include Synechococcus sp., Chlorococcales sp. and Volvocales sp., among others.

The aeration, temperature, pH, nutritional requirements, intensity and wavelength of light (e.g., white light from natural or artificial light source), as well as duration of light/dark cycles suitable to support the growth and metabolism of photosynthetic microorganisms are known, for example, and are described in U.S. Pat. No. 7,371,560 which is hereby incorporated by reference.

### Acclimation and Conditions

The bioreactor(s) are inoculated with the selected microorganism or mixed culture, followed by an acclimation period. The acclimation period may last one month, two weeks, one week, or less, during which biofilms are formed on support surfaces and the desired metabolic processes induced.

Acclimation of the microorganisms can be determined by a decrease in the lag period of activity upon introduction of the substrate or an increase in degradation or synthesis rate. Acclimation may involve, for example, induction or derepression of enzymes, multiplication of the initially small population(s) of degrading or synthetic microorganisms, selection for beneficial mutations, optimization of inorganic nutrients or other conditions, depletion of alternative carbon sources that may be present, adaptation of microorganisms to toxins or inhibitors that may be present, and predation by certain microorganisms (e.g., protozoa).

Acclimation in some embodiments may proceed in steps, by first acclimating xenobiotic-degrading bacteria for the xenobiotic carbon source, followed by acclimating biosynthetic microorganisms for the production of the desired product(s). When using an integrated system as described herein, the flow of substrate material from the aerobic/anaerobic biodegradation reactors to the anaerobic biosynthesis reactor can be controlled or initiated once degrading bacteria are fully or sufficiently acclimated. Such embodiments may be useful particularly where the xenobiotic is toxic or is degraded to components that are toxic to biosynthetic microbes.

During the acclimation period, it may be important to limit the concentration of the xenobiotic compound and/or the flow of substrate through the system. When a multiphasic system is employed, growth and selection of xenobiotic-degrading microorganisms may be evaluated on the basis of the activity of adhering biomass (forming a biofilm) on the solid surfaces as well as at liquid interfacial areas that may be present. Growth and selection of biofuel-synthesizing microorganisms may be evaluated by the appearance and concentration of product being produced, as well as by the production of the expected metabolites (e.g., CO₂, methane, ethanol, butanol, hydrogen, etc.).

During acclimation and after acclimation, the bioreactor conditions may be adjusted as necessary to optimize product yield and rate of synthesis. Such conditions include xenobiotic input concentration, flow rate, bioreactor temperature(s), levels of bioreactor agitation, and levels of aeration or oxygenation. For example, the temperature of biodegradation and fermentative reactors may be maintained within the range of about 15° C. to about 35° C., such as about 25 to about 30° C. The flow-rate of substrate through the system may depend on the volume of the bioreactor, and the degradation rate, and may be maintained by a system of pumps as described in more detail herein. The bioreactor may further allow for agitation of the medium, if necessary to maintain the availability of nutrients. Degradation of the xenobiotic may be monitored by sampling the xenobiotic-containing phase or by monitoring CO₂ and/or biomass production.

In certain embodiments, complete decomposition of the xenobiotic substrate may take place first, independently of biofuel synthesis, or alternatively, biosynthesis may take place simultaneously with decomposition in a coupled decomposition/synthesis bioreactor. In certain embodiments that employ independent decomposition and biosynthesis reactors, the decomposition process may proceed for from about 1 hour to about 1 week, or about 10 hours to about 3 days, or in certain embodiments, for about 1, about 2, about 3, about 4, or about 5 days. For example, xenobiotic degradation may proceed for (be substantially complete at) about 3 hours, about 5 hours, about 10 hours, about 15 hours, or about 24 hours. Alternatively, a coupled/integrated biodegradation and biosynthesis system may convert xenobiotic compounds to biofuel in about 1 week or less, about 4 days or less, about 2 days or less, about 1 day or less, about 15 hours or less, or about 10 hours or less. The length of time needed for the bioprocesses will depend on several conditions, including, xenobiotic/biomass input concentration, flow rate, volume of bioreactors, bioreactor temperature(s), levels of bioreactor agitation, and levels of aeration or oxygenation.

In certain embodiments, biofuel or bioenergy production is at an industrial scale with a continuous or semi-continuous integrated biodegradation/biosynthesis system, such that from about 100 to about 100,000 gallons of xenobiotic-containing substrate are degraded per day. For example, about 500 to about 10,000 gallons of xenobiotic-containing substrate may be processed to biomass and converted to biofuel in a period of about 24 hours to about 48 hours. In certain embodiments, about 500 to about 10,000 gallons of xenobiotic-containing substrate may be processed to biomass and converted to biofuel in a period of less than about 24 hours.

Recovery of Biofuel Products

Biofuel products may be recovered and/or purified by known and commercially available methods and devices.

Alcohols such as ethanol, methanol, and/or butanol may be recovered from liquid material by molecular sieves, distillation, and/or other separation techniques. For example, ethanol can be concentrated by fractional distillation to about 90% or about 95% by weight. There are several methods available to further purify ethanol beyond the limits of distillation, and these include drying (e.g., with calcium oxide or rocksalt), the addition of small quantities of benzene or cyclohexane, molecular sieve, membrane, or by pressure reduction.

Product gas, for example, as produced by anaerobic metabolism or photosynthesis, may be processed to separate the methane and/or hydrogen components. Methane, hydrogen, or biogas may be drawn off from the system as pipeline gas.

In accordance with the invention, methane and/or hydrogen may be recovered as a biofuel product. Methane may be recovered and/or purified from biogas by known methods and systems which are commercially available, including membrane systems known for separating gases on the basis of different permeabilities. See, for example, U.S. Pat. No. 6,601,543, which is hereby incorporated by reference. Alternatively, various methods of adsorption may be used for separating methane and hydrogen.

Other ways of collecting biofuel products including centrifugation, temperature fractionalization, chromatographic methods and electrophoretic methods.

In certain embodiments, the biofuel recovery/purification components may be integrated into the system, for example, by connecting the respective device or apparatus to the gas or liquid effluents from the biosynthetic bioreactors. The purified biofuels and bioenergy products may be stoked in a separate container(s).

### Integrated Systems for Converting Xenobiotics to Biofuel

The present invention further provides an integrated system for generating biofuels, such as hydrogen and methane, as well as other useful products such as ethanol, butanol, methanol, and biodiesel. Exemplary integrated systems are illustrated in FIG. 2 and FIG. 3.

The system comprises one or more multiphasic bioreactors suitable for breaking down toxic material by aerobic, anaerobic, and/or anoxic microbial processes, thereby producing biomass. The bioreactor for decomposition comprises an inlet for influent waste (xenobiotic liquid) to the bioreactor system. The bioreactor for decomposition further comprises an inlet for oxygenation of the aerobic bioreactor. The anaerobic and aerobic chambers may be connected so as to allow recirculation of liquid substrate. Recirculation allows the exchange of metabolites and cells between the aerobic and anaerobic chambers, providing for their cooperative metabolism. Coupled aerobic/anaerobic reactors have been described herein.

The system may comprise a multiphasic, coupled, aerobic-anaerobic reactor system essentially as illustrated in FIG. 2 (left-hand side). This system allows for substrate to circulate between the anaerobic and aerobic decomposition reactors, and may comprise a feedback port for feeding recycled, partially metabolized compounds from the biosynthesis reactor(s) back for further biodegradation.

The decomposition bioreactor further comprises an outlet for effluent liquid from the biodegradation system, such liquid comprises metabolized and partially metabolized substrate, as well as cells and cellular debris, to act as carbon sources for the biosynthesis of biofuel products. The anaerobic biosynthesis reactor(s) comprise an inlet for this liquid waste circulating from the multiphasic degradation system.

The biodegradation system further comprises an outlet for effluent gas produced as a result of catabolism in the anaerobic biodegradation system. The gas effluent, which comprises significant amounts of CO₂, is fed to the photo bioreactor to support the metabolism of photosynthetic microorganisms (as described below).

The biosynthesis system may be an in-series anaerobic/photosynthesis bioreactor system. In these embodiments, the photo reactor(s) comprise an inlet for gas waste from the anaerobic biosynthesis reactor, as well as in some embodiments the anaerobic degradation reactor.

The biosynthesis reactors further comprise an outlet from the anaerobic chamber to transport biofuels and bioenergy products (e.g., methane or alcohols), and an outlet from the photo bioreactor to transport biofuels and bioenergy products (e.g., H₂ or lipids). The system may further comprise a mechanism for recovery of products obtained by the biosynthetic processes, and a container or feed to recover non-fuel compounds for feedback to biodegradation. Thus, the system may comprise a feedback connection between the biosynthesis system and the biodegradation system to recycle all materials not completely used. In such embodiments, the present invention provides a “zero pollution system” preventing accumulation of virtually any significant pollutants while achieving substantially complete mineralization with biofuel/bioenergy production.

The aerobic bioreactors are equipped with an aeration system(s), to allow for injection of oxygen-containing gas into the bioreactor. In certain embodiments, the oxygen-containing gas is one or more of air, oxygen and hydrogen peroxide. The amount of oxygen dissolved in the waste liquid may vary, but generally approximates the rate of oxygen-consumption of the aerobic biofilm.

A pump system may be employed for connecting inlets and outlets, to and from bioreactors, to maintain the desired flow through the system. Valves may also be present to allow for the flow of material through the system to be controlled.

The system may include sampling ports, so that biodegradation of the xenobiotic and/or biofuel production can be monitored, as well as oxygen consumption, CO₂ production, pH, oxidation-reduction conditions, microbial cell physiology, biofilm health, among others.

The integrated system is generally a continuous system, with material circulating between the biodegradation and biosynthesis reactors, as controlled by the system of pumps. For example, in the continuous system, the anaerobic biosynthesis reactor may operate with an incoming flow rate equal to the outlet flow rate of liquid from the biodegradation reactor. Further, the photo reactor may operate with a gas influent that is equivalent to the effluent gas of the anaerobic reactors.

In certain embodiments, as illustrated in FIG. 3, the integrated system is a coupled multiphasic biodegradation system and a multiphasic biosynthesis system. In these embodiments, multiphasic bioreactor system for xenobiotic biodegradation comprises an aerobic multiphasic bioreactor and an anaerobic multiphasic bioreactor. The multiphasic reactor system for biofuels and bioenergy production comprises a multiphasic anaerobic bioreactor for biosynthesis, and a multiphasic photo bioreactor for biosynthesis. Multiphasic bioreactors have been described (see FIG. 4).

The working volume of the system may be from about 100 gallons to about 100,000 gallons. For example, the industrial system may convert from about 500 to about 10,000 gallons of source material to useful product per run (in batch), or per day (when continuous). The operation or retention time for converting product to fuel may be about 2 weeks or less, but in some embodiments is 1 week or less, such as 3 days, 2 days, or 1 day or less (e.g., 15 hours).

In certain embodiments, the system is configured to allow for the transportation of biomass and/or biogas produced at one location (by a biodegradation reactor as described herein), to be transported to another location for synthesizing biofuel or bioenergy products (by a biosynthesis bioreactor as described herein). Alternatively, the system may be entirely integrated to couple the biodegradation of xenobiotic compounds with the biosynthesis of bioenergy products from resulting biomass and biogas. The system (or the biodegradation components) may be connected to, or positioned or located near, the production or source of such xenobiotic compounds, so as to obviate the need to transport the waste, which may be hazardous or toxic, for disposal. For example, the system or biodegradation components may be located within about 1 mile or less of the production or source of the xenobiotic compound.

### EXAMPLES

### Example 1

### Coupled Biodegradation/Biosynthesis System for the Production of Methane from Recalcitrant Xenobiotic

To demonstrate that a xenobiotic recalcitrant compound can be transformed to usable biofuel products by aerobic and anaerobic biofilm bacteria cultured in multiphasic bioreactors, 3,4-Diclhorobenzoic acid (3,4-DCB) was processed using the reactor shown diagrammatically in FIG. 2 (“BIODSYNT”). 3,4-DCB is a recalcitrant xenobiotic in the environment, produced and accumulated by the partial degradation of Polychlorobiphenyls (PCBs) and other herbicides.

Bacteria attached to liquid or solid surfaces are efficient for breaking down xenobiotic compounds. Further, while aerobic and anaerobic bacteria may each have limitations in breaking down a xenobiotic compound, these limitations can be overcome by their cooperative metabolisms, that is, between both aerobic and anaerobic biofilm bacteria in a multiphasic system. This in turn permits the complete mineralization of recalcitrant xenobiotic compounds.

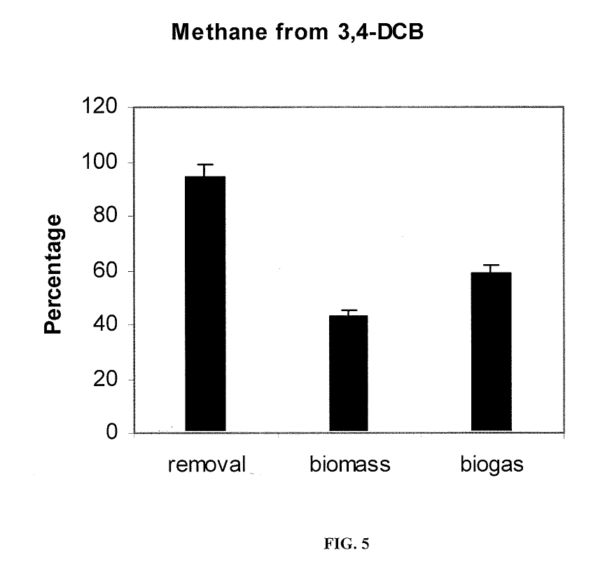

The biodegradation reactors (FIG. 2) were fed with 500 to 1000 mg/l of the xenobiotic. The results of 3,4-DCB biodegradation are shown in FIG. 5. At the hydraulic retention time of 16 hours, the xenobiotic disappearance (removal) in the system was nearly 100%. The biomass yield in both anaerobic and aerobic reactors, determined from suspended produced cells was close to the theoretically expected value of 43%.

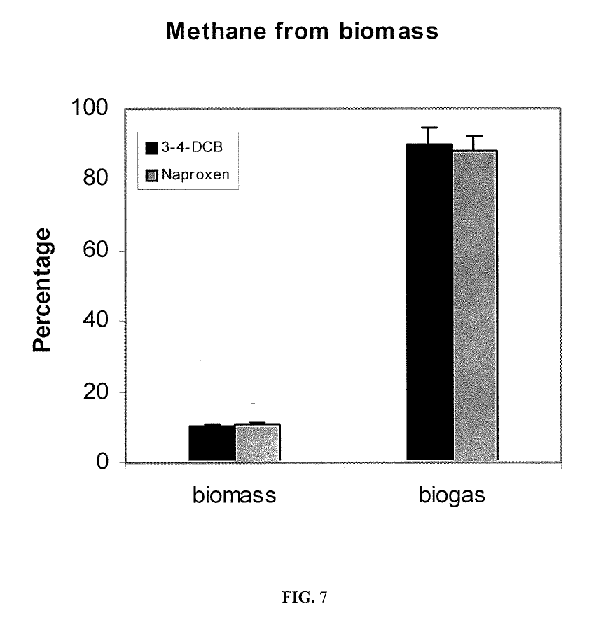

The production of methane in the anaerobic biosynthesis reactor (far right AN reactor in FIG. 2) was determined. In the anaerobic biosynthesis system, at 16 hours of retention time the methanogenic cultures (consortium) converted about 60% of the available substrate to biogas (FIG. 5), while at 20 hours of retention time the methane production reached 90% and biomass was almost completely consumed (FIG. 7). The biogas produced contained about 55% CH₄ and 44% CO₂, which were close to the theoretically expected values.

These results indicate that the BIODSYNT system was highly efficient in the degradation, mineralization, and bioconversion of 3,4-DCB to bioenergy products. These results demonstrate that the coupled biodegradation/biosynthetic system can be used to produce bioenergy and biofuels using, as raw materials, recalcitrant xenobiotic compounds disposed by industry.

### Example 2

### Coupled Biodegradation/Biosynthesis System for the Production of Biofuels from Pharmaceutical Compound

As a pharmaceutical model (xenobiotic) compound, Naproxen ((+)-(S)-2-(6-methoxynaphthalen-2-yl) was used as a carbon source for the production of biofuel, and particularly methane, using the BIODSYNT system (FIG. 2, and Example 1, above). Naproxen is a non-steroidal anti-inflammatory drug (NSAID) commonly used for the reduction of moderate to severe pain, fever, inflammation and stiffness caused by a variety of conditions. Naproxen and naproxen sodium are marketed under various trade names including: XENOBID, ALEVE, ANAPROX, MIRANAX, NAPROGESIC, NAPROSYN, NAPRELAN, PROXEN, and SYNFLEX.

Using the biofim multiphasic bioreactors of Example 1, the aerobic/anaerobic bacteria were acclimated by feeding the xenobiotic in low concentrations (about 30 mg/l) to the degradation bioreactor system. The acclimation proceeded for about 2 weeks until an increase in suspended biomass was observed in the outlet liquid of the biodegradation system. At the end of acclimation phase, the removal of the Naproxen was evaluated in the biodegradation system. The removal efficiency of the degradation reactors was close to 98%.

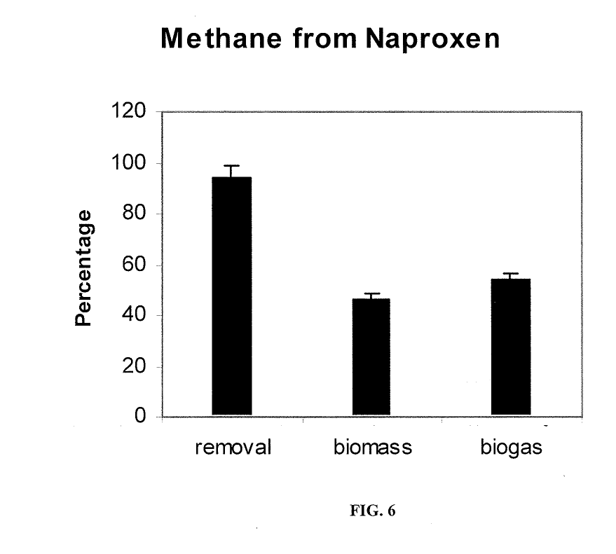

The concentration of the xenobiotic was increased weekly for 4 weeks up to a concentration of 500 mg/l in the biodegradation reactors. As shown in FIG. 6, the anaerobic synthetic reactor showed that 97% of the Naproxen was removed during 30 days of continuous culture. Increasing the concentration of Naproxen over 500 mg/l slowed the removal of the compound. The biomass production was around 41% of the pharmaceutical carbon source (FIG. 6). The total biogas production at 16 hours of retention time in the anaerobic biosynthetic reactor reached 54% of the degraded pharmaceutical compound (FIG. 6), while at 20 hours of retention time the biogas reached about 90%.

The reactor's performance for the treatment of Naproxen was evaluated at a 10 hour retention times. Decreasing the retention times of the anaerobic bioreactor from 16 to 10 hours did not impact biomass consumption or biogas production.

These results demonstrate that the BIODSYNT system efficiently converts Naproxen to bioenergy compounds, such as methane.

### Example 3

### Photo Bioreactor for the Production Hydrogen and Biodiesel from CO₂ Effluents

The BIODSYNT system (FIG. 2) was tested for its ability to fix and use CO₂, through the cultivation of a blue-green algae (cyanobacteria) in a photosynthetic bioreactor.

A culture of cyanobacterium Synechococcus sp. was inoculated in a 1 L photo bioreactor containing a basic mineral salts medium. The reactor was incubated at room temperature under 1.2 klux light intensity and a 16:8 h light:dark cycle. The cultures were subjected to CO₂ at 0.5 and 1.0% (v/v) levels. The growth of culture was recorded at 5-day intervals.

Synechococcus sp. was evaluated for biomass growth, lipid content and hydrogen production. As showed in FIG. 7, more biomass was achieved at 1% CO₂ than at 0.5%. In both cases, the lipids extracted to produce biodiesel was approximately 50% of the biomass. Around 150 nmol of hydrogen per gram of protein was observed when biomass was grown at 1% CO₂. About half that amount was produced with 0.5% CO₂.

To demonstrate that effluent gas produced in the bioreactors can be captured before leaving the BIODSYNT system, the gas effluent of the anaerobic bioreactors was connected in line with a photo bioreactor. As in the tests above, the waste gas from the anaerobic bioreactor was able to support the growth of Synechococcus sp. Thus, the CO₂ effluent from the anaerobic bioreactor can be captured to support the growth and metabolism of blue-green algae cells in a photo bioreactor system. This photo bioreactor can be used to capture the CO₂ produced in the BIODSYNT aerobic and anaerobic reactors.

The photo bioreactor can be used to capture the CO₂ produced in the BIODSYNT aerobic and anaerobic reactors.

### REFERENCES

The following references are hereby incorporated by reference in their entireties.
Ascon M A, Ascon D B, Lebeault J M. (1995). Degradation activity of adhered and suspended Pseudomonas cells cultured on 2,4,6-trichlorophenol, measured by indirect conductimetry. J Appl Bacteriol. 79:617-624.
Ascon M A, Thomas D, Lebeault J M. (1995). Activity of synchronized cells of a steady state biofilm recirculated reactor during xenobiotic biodegradation. Appl. Environ. Microbiol. 61(3):920-5.
Ascon M A, Lebeault J M. (1995). Interfacial area effects of a biphasic aqueous/organic system on growth kinetic of xenobiotic-degrading microorganisms. Appl. Microbiol. Biotechnol. 43:1136-1141.
Ascon M A, Lebeault J M. (1999). High Efficiency of a coupled aerobic-anaerobic recycling biofilm reactor system in the degradation of recalcitrant chloroaromatic xenobiotic compounds. Appl. Microbiol. Biotechnol. 52(4):592-9.
Ascon M A, Lebeault J M. (1993). Selection of Xenobiotic-degrading microorganisms in a biphasic aqueous-organic system. Appl. Environ. Microbiol. 59(6):1717-1724.
Aust S D, Bouquin A, Loper J C, Salanitro J P, Suk W A. (1994). Biodegradation of Hazardous Wastes. Environmental Health Perspectives Supplements 102 (Suppl. 1):245-252.
Janssen D B, Dinkla I J T, Poelarends G J, Terpstra P (2005). Bacterial degradation of xenobiotic compounds: evolution and distribution of novel enzyme activities. Environ. Microbiol. 7(12)1868-1882.
Kastner M. (1991). Reductive dechlorination of tri-and tetra-chloroethylenes depends on transition from aerobic to anaerobic conditions. Appl Environ Microbiol 57: 2039-2046
Armenante P M, Kafkewitz D, Lewandowski G, Kung C M. (1992). Integrated anaerobic-aerobic process for the biodegradation of chlorinated aromatic compounds. Environ Prog 11: 113-122
Dienemann E A, Kosson D S, Ahlert R C. (1990). Evaluation of serial anaerobic/aerobic packed bed bioreactors for treatment of a superfund leachate. J Hazard Mater 23: 21-42
Fathepure B Z, Vogel T M. (1991). Complete degradation of polychlorinated hydrocarbons by a two-stage biofilm reactor. Appl Environ Microbiol 57: 3418±3422
Okubo T, Okada M, Murakami A, Inamori Y. (1990). Influence of daily variation of flow and pollution load on the performance of submerged anaerobic/aerobic biofilm system. Water Sci Technol 22: 153-160
Fahmy M, Heinzle E, Kut O M. (1991). Treatment of bleaching effuents in aerobic/anaerobic fluidized biofilm systems. Water Sci Technol 24: 179-187

1. An integrated system for converting a xenobiotic substrate to a biofuel product, comprising:
one or more first bioreactors suitable for decomposing the xenobiotic substrate by microbial action, to thereby produce biomass and/or biogas, and
one or more second bioreactors operably connected to said one or more first bioreactors to receive the biomass and/or the biogas, wherein the one or more second bioreactors are suitable for synthesizing one or more biofuels products from the biomass and/or the biogas by microbial action.

2. The integrated system of claim 1, wherein the one or more first bioreactors includes an aerobic bioreactor and an anaerobic bioreactor.

3. The integrated system of claim 2, wherein the aerobic bioreactor includes an inlet for oxygenation of the aerobic bioreactor.

4. The integrated system of claim 2, wherein the aerobic bioreactor and the anaerobic bioreactor are one of an aerobic-anaerobic recycle reactor system and an in-series anaerobic-aerobic reactor system.

5. The integrated system of claim 2, wherein the aerobic bioreactor and the anaerobic bioreactor are an aerobic-anaerobic recycle reactor system that allows circulation of liquid xenobiotic substrate between the anaerobic and aerobic bioreactors.

6. The integrated system of claim 1, wherein the one or more first bioreactors are multiphasic bioreactors containing xenobiotic-degrading microorganisms forming biofilms.

7. The integrated system of claim 6, wherein the biofilms are attached to a solid support matrix.

8. The integrated system of claim 6, wherein biofilms are further attached to a liquid surface.

9. The integrated system of claim 6, wherein the anaerobic bioreactor contains anaerobic and/or facultative xenobiotic-degrading microorganisms forming biofilms.

10. The integrated system of claim 6, wherein the aerobic bioreactor contains aerobic and/or facultative xenobiotic-degrading microorganisms within biofilms.

11. The integrated system of claim 9, wherein the xenobiotic-degrading microorganisms are one or a consortium of microorganisms listed in Table 2.

12. The integrated system of claim 11, wherein the xenobiotic-degrading microorganisms include one or more of a Pseudomonas sp., an Arthrobacter sp., an Acynebacter sp., and an Alcaligenes sp.

13. The integrated system of claim 1, wherein the one or more bioreactors for decomposing xenobiotic substrate includes an inlet for introducing xenobiotic-containing liquid into the system.

14. The integrated system of claim 3, wherein the inlet for oxygenation is connected to a source of oxygen-containing gas.

15. The integrated system of claim 14, wherein the oxygen-containing gas is one or more of air, oxygen and hydrogen peroxide.

16. The integrated system of claim 1, wherein the one or more second bioreactors include at least one anaerobic bioreactor configured to produce a biofuel product from the biomass by fermentation.

17. The integrated system of claim 16, wherein the biofuel product is a low molecular weight alcohol.

18. The integrated system of claim 16, wherein the biofuel product includes one or more of methanol, ethanol, and butanol.

19. The integrated system of claim 16, wherein the one or more second bioreactors include one or a consortium of yeasts.

20. The integrated system of claim 19, wherein the yeasts include one or more of a Saccharomyces sp., a Klyveromyces sp., a Candida sp., and a Pichia sp.

21. The integrated system of claim 16, wherein the one or more second bioreactors include one or more, or a consortium of bacteria.

22. The integrated system of claim 21, wherein at least one bacteria is a Zymomonas sp. a Erwinia sp., a Serratia sp., a Lactobacillus sp., a Lactococcus sp., a Clostridium sp., and a Acetobacter sp.

23. The integrated system of claim 22, wherein the one or more second bioreactors include at least one bioreactor for producing methane from the biomass by methanogenesis.

24. The integrated system of claim 23, wherein the methanogen is one or more, or a consortium of Methanobacterium sp., Methanothrix sp., Methanosarcina sp., and Methanomonas sp.

25. The integrated system of claim 16, wherein the one or more second bioreactors are multiphasic bioreactors containing biofuel-synthesizing microorganisms forming biofilms.

26. The integrated system of claim 25, wherein the biofilms are attached to solid and/or liquid support surfaces.

27. The integrated system of claim 1, further comprising at least one photo bioreactor configured to convert carbon dioxide in the biogas to at least one biofuel product by photosynthesis.

28. The integrated system of claim 27, wherein the photo bioreactor includes one or more, or a consortium of algae(s).

29. The integrated system of claim 28, wherein the algae(s) are one or more of a green algae, a blue-green algae and/or a red algae.

30. The integrated system of claim 29, wherein at least one algae is a Synechococcus sp., a Chlorella sp., a Synechocystis sp., a Nitzchia sp., and/or a Schizochytriu sp.

31. The integrated system of claim 27, wherein the photo bioreactor is configured to produce hydrogen gas and/or lipids.

32. The integrated system of claim 27, wherein the one or more first bioreactors includes an anaerobic bioreactor having an outlet for effluent biogas, wherein the effluent biogas can be fed to the photo bioreactor as a carbon source.

33. The integrated system of claim 27, wherein the one or more second bioreactors for biosynthesis includes an anaerobic bioreactor having an outlet for effluent biogas, wherein the effluent biogas can be fed to the photo bioreactor as a carbon source.

34. The integrated system of claim 1, further comprising a mechanism for collecting and/or recovering the biofuel or bioenergy product.

35. The integrated system of claim 34, wherein the mechanism is a molecular sieve, distillation system, and/or semi-permeable membrane.

36. The integrated system of claim 1, further comprising a feedback connection between the one or more second bioreactors and the one or more first bioreactors, so as to continuously recycle liquid material not converted to biofuel product.

37. The integrated system of claim 34, further comprising an outlet to transport methane or alcohols.

38. The integrated system of claim 34, further comprising an outlet to transport hydrogen gas and/or lipids.

39. The integrated system of claim 1, further comprising a system of pumps and/or valves connecting inlets and outlets between the one or more first bioreactors and the one or more second bioreactors.

40. The integrated system of claim 39, wherein the pumps and/or valves control the flow of liquid and/or gas through the system.

41. The integrated system of claim 1, further comprising one or more sampling ports to monitor one or more of xenobiotic concentration, oxygen consumption, pH, and CO₂ production.

42. The integrated system of claim 1, wherein the working volume of the system is from about 100 gallons to about 100,000 gallons.

43. The integrated system of claim 1, wherein the working volume of the system is from about 500 gallons to about 50,000 gallons.

44. The integrated system of claim 1, wherein the system is located within 1 mile of the source of said xenobiotic substrate.

45. A method for producing one or more biofuel products from a xenobiotic substrate, comprising:
decomposing the xenobiotic substrate by microbial action to produce biomass and/or biogas, and
synthesizing one or more biofuel products from the biomass and/or biogas by fermentative, methanogenic, and/or photosynthetic microorganisms.

46. The method of claim 45, wherein said xenobiotic substrate is an aliphatic or aromatic hydrocarbon.

47. The method of claim 45, wherein the xenobiotic substrate is a halogenated hydrocarbon.

48. The method of claim 45, wherein the xenobiotic substrate is a heteroaromatic compound.

49. The method of claim 45, wherein the xenobiotic substrate is a dioxin, furan, or polychlorinated biphenyl.

50. The method of claim 45, wherein the xenobiotic substrate is one or more of a pharmaceutical, pharmaceutical byproduct, cosmetic, personal care product, or pesticide.

51. The method of claim 50, wherein the xenobiotic substrate is fossil fuel pollution.

52. The method of claim 45, wherein the xenobiotic substrate is a polycyclic aromatic hydrocarbon.

53. The method of claim 45, wherein the xenobiotic substrate is soluble in an aqueous phase.

54. The method of claim 53, wherein the xenobiotic substrate is insoluble in an aqueous phase.

55. The method of claim 45, wherein the xenobiotic substrate is decomposed by circulating the xenobiotic substrate between one or more aerobic and one or more anaerobic bioreactors.

56. The method of claim 55, wherein the one or more aerobic and one or more anaerobic bioreactors are multiphasic bioreactors.

57. The method of claim 45, wherein the xenobiotic-degrading microorganisms are one or a consortium of microorganisms listed in Table 2.

58. The method of claim 56, wherein the multiphasic bioreactors harbor the xenobiotic-degrading microorganisms on solid support matrices within biofilms.

59. The method of claim 55, wherein a bioreactor for synthesizing one or more biofuel products is operably connected to receive biomass from the aerobic bioreactor.

60. The method of claim 59, wherein the bioreactor for synthesizing one or more biofuel products is an anaerobic fermentation bioreactor.

61. The method of claim 60, wherein the biofuel product is a small molecular weight alcohol.

62. The method of claim 61, wherein the biofuel product is ethanol, methanol, and/or butanol.

63. The method of claim 60, wherein the one or more biofuel products are synthesized by one or a consortium of yeasts.

64. The method of claim 63, wherein the yeasts include one or more of a Saccharomyces sp., a Klyveromyces sp., a Candida sp., and a Pichia sp.

65. The method of claim 60, wherein the one or more biofuel products are synthesized by one or a consortium of bacteria.

66. The method of claim 59, wherein the bioreactor for synthesizing one or more biofuel products is a methanogenesis bioreactor.

67. The method of claim 66, wherein the methanogenesis bioreactor comprises one or a consortium of Methanobacterium sp., Methanothrix sp., Methanosarcina sp., and Methanomonas sp.

68. The method of claim 59, wherein the bioreactor for synthesizing one or more biofuel products is a multiphasic bioreactor containing biofuel-synthesizing microorganisms forming biofilms.

69. The method of claim 68, wherein the biofilms are attached to solid support matrices.

70. The method of claim 55, wherein a photo bioreactor is operably connected to receive the biogas containing carbon dioxide from the anaerobic degradation reactor(s).

71. The method of claim 70, wherein the photo bioreactor is further operably connected to receive biogas containing carbon dioxide from an anaerobic fermentation bioreactor.

72. The method of claim 70, wherein the photo bioreactor comprises one or more, or a consortium of algae(s).

73. The method of claim 72, wherein the algae(s) are one or more of a green algae, a blue-green algae and/or a red algae.

74. The method of claim 73, wherein at least one algae is a Synechococcus sp., a Chlorella sp., a Synechocystis sp., a Nitzchia sp., and/or a Schizochytriu sp.

75. The method of claim 70, wherein the photo bioreactor produces hydrogen gas and/or lipids as biofuel products.

76. The method of claim 45, further comprising, recovering or purifying the one or more biofuel products.

77. The method of claim 76, wherein the biofuel product(s) are recovered or purified by a molecular sieve, distillation system, and/or semi-permeable membrane.

78. The method of claim 45, further comprising, returning non-fuel products from the one or more second bioreactors back to the one or more first bioreactors for further bioprocessing.
